SEQUENCE LISTING (8 sequences)

SEQ ID NO: 1 - DNA, 28 bp, Artificial sequence
  primer
tacactcaacaaagatcaacttctgtca

SEQ ID NO: 2 - DNA, 27 bp, Artificial sequence
  primer
catgccacataacttattgatgtgttt

SEQ ID NO: 3 - DNA, 26 bp, Artificial sequence
  primer
cattaaataaggatcagctgctgtca

SEQ ID NO: 4 - DNA, 28 bp, Artificial sequence
  primer
gcataccacatagtttgtttaggtgttt

SEQ ID NO: 5 - DNA, 24 bp, Artificial sequence
  probe
caccatccaacggagcacaggaga

SEQ ID NO: 6 - DNA, 30 bp, Artificial sequence
  probe
ataatattgacactcccaattatgatgtgc

SEQ ID NO: 7 - DNA, 121 bp, Artificial sequence
  amplicon
tacactcaacaaagatcaacttctgtcatccagcaaatacaccatccaacggagcacaggagatagtattgatactcctaattatgatgtgcagaaacacatcaataagttatgtggcatg

SEQ ID NO: 8 - DNA, 120 bp, Artificial sequence
  amplicon
cattaaataaggatcagctgctgtcatccagcaaatacactattcaacgtagtacaggagataatattgacactcccaattatgatgtgcaaaaacacctaaacaaactatgtggtatgc

(19) United States
(12) Patent Application Publication
(10) Pub. No.: US 2007/0065813 A1
(43) Pub. Date: Mar. 22, 2007

(54) Assay for quantitative detection of respiratory syncytial virus (RSV)

(76) Inventors: Randall T. Hayden, Memphis, TN (US); Zhengming Gu, Cordova, TN (US)

Correspondence Address: Licata & Tyrrell P.C., 66 E. Main Street, Marlton, NJ, 08053, US

(21) Appl. No.: 11/230,963

(22) Filed: Sep. 20, 2005

(51) Int. Cl. C12Q 1/70; C12Q 1/68; C07H 21/04

(52) U.S. Cl. 435/5; 435/6; 536/24.1

(57) ABSTRACT

The present invention describes respiratory syncytial virus (RSV) primers and probes as reagents for detection of a RSV infection. Specifically, the invention provides nucleic acid sequences from the RSV nucleocapsid gene used with hybridization and PCR techniques to detect RSV infection.

### FIELD OF THE INVENTION

This invention relates to the detection of respiratory syncytial virus (RSV) using a real-time reverse transcription polymerase chain reaction (RT PCR) assay.

### BACKGROUND OF THE INVENTION

Respiratory syncytial virus (RSV) is a negative-strand RNA virus belonging to the family, Paramyxoviridae and to the genus, pneumovirus. The structure and composition of RSV have been described in detail. (Domawchowske et al., Clinical Microbiology Review 12:298-309 (1999)). Two major subgroups of RSV, type A and B, have been identified, as well as antigenic variants within each subgroup (Anderson; L. J. et al., J Inf Dis 151:626-633 (1985); Mufson, M. A. et al., J Gen Virol 66:2111-2124 (1985)).

RSV is a major human pathogen, responsible for respiratory infection in patients of all ages. Typically, RSV infections remain localized to the upper respiratory tract, causing profuse rhinorrhea, nasal congestion, pharyngitis, cough and fever. In some patients, however, infection spreads to the lower respiratory tract. Severe lower respiratory tract disease (e.g., pneumonia, bronchiolitis) results, and typically requires hospitalization and breathing support. In some circumstances, RSV may be fatal.

Infants and young children, as well as immunocompromised patients and the elderly, are particularly at risk for serious respiratory illness related to RSV infection. Premature infants, as well as young children with chronic heart and lung disease, are at greatest risk for serious complications from RSV infection. Yet, 75% of the hospitalizations for RSV infection occur in infants and children that were previously healthy and without risk factors other than age. Approximately 100,000 children are hospitalized annually in the U.S. with severe cases of pneumonia and bronchiolitis resulting from an RSV infection (Hall, C. B. et al., N Engl J Med 344:1917-1928 (2001)). Moreover, RSV is responsible for hundreds of deaths in infants and young children each year.

RSV is highly contagious. Spread person to person through infected nasal and oral secretions, RSV can also live on surfaces for hours. Typically, RSV occurs in epidemics that last up to 4 months, from late fall through early spring. Children in day-care centers and preschools are at significant risk, and the elderly in hospitals and nursing homes are particularly vulnerable. School-aged children are commonly implicated in the spread of the disease, both to their younger siblings and their parents.

It has been recently reported that RSV accounts for 13.5-21.6% of the $2.25 billion in costs associated with hospitalization of infants with lower respiratory infections. The total costs are even greater, as these include treatment for other populations at high risk for RSV, including the elderly and the immunocompromised. RSV also affects healthy adults, and even in a milder form is associated with significant work absences. Re-infection with RSV is very common, forcing additional costs on the healthcare system. The health-related effects of initial infection in some populations, moreover, may be long term. Recent data indicates that RSV-induced lower respiratory tract infections in infants may be linked to the development of asthma or reactive airway disease in later childhood (Sigurs, N. et al., Am J Resp Crit Care Med 161:1501-1507 (2000)).

Current diagnostic methods include culture, immunofluoresence, and enzyme-linked immunoassay, which can be slow and non-specific. In light of the fact that RSV remains a serious public health threat, there remains a strong need to provide a highly sensitive, rapid and easy assay for diagnosing the disease. It is therefore an object of the present invention to provide methods for the diagnosis of human patients infected with RSV.

### SUMMARY OF THE INVENTION

An aspect of this invention is directed to oligonucleotide primers and probes optimized for the detection of respiratory syncytial virus (RSV). In particular oligonucleotide primers having the sequences set forth in SEQ ID NOS: 1-4 and oligonucleotide probes having the sequences set forth in SEQ ID NOS: 5-6 are provided.

Another aspect of this invention is directed to a method for detecting the presence or absence of RSV using one or more of the oligonucleotide primers and probes provided herein. The method can be used to detect RSV in a patient or in a sample of interest. The method is preferably based on a real-time reverse transcription polymerase chain reaction (RT PCR). The invention also includes kits and sets of one or more of the oligonucleotides provided herein useful in the performance of this method.

### DESCRIPTION OF THE SEQUENCE LISTINGS

SEQ ID NO: 1 is an oligonucleotide that acts as a forward primer in PCR to amplify a portion of the nucleocapsid gene in RSV A.

SEQ ID NO: 2 is an oligonucleotide that acts as a reverse primer in PCR to amplify a portion of the nucleocapsid gene in RSV A.

SEQ ID NO: 3 is an oligonucleotide that acts as a forward primer in PCR to amplify a portion of the nucleocapsid gene in RSV B.

SEQ ID NO: 4 is an oligonucleotide that acts as a reverse primer in PCR to amplify a portion of the nucleocapsid gene in RSV B.

SEQ ID NO: 5 is a nucleotide probe that hybridizes to a portion of the nucleocapsid gene in RSV A.

SEQ ID NO: 6 is a nucleotide probe that hybridizes to a portion of the nucleocapsid gene in RSV B.

### DETAILED DESCRIPTION OF THE INVENTION

The invention is directed toward methods that detect a broad range of RSV strains, including both subgroups A and B. The invention is further directed to a method of detecting the presence or absence of RSV in a patient, said method comprising (a) preparing a nucleic acid sample from the patient suitable for use in a RT PCR assay, (b) forming a RT PCR solution containing at least a portion of said nucleic acid sample from step (a), a RT PCR primer and probe set comprising one or more oligonucleotides having the sequences set forth in SEQ ID NOS: 1-6, a mixture of nucleoside triphosphate monomers, and an enzyme Taq polymerase in a buffered solution, (c) carrying out a RT PCR reaction on the solution formed in step (b); and (d) detecting the presence or absence of RT PCR products formed from said solution, wherein the presence of RT PCR products formed by the amplification of RSV nucleic acid indicates the presence of RSV in the patient and the absence of said RT PCR products indicates the absence of RSV in the patient. In a preferred embodiment, the method uses a primer and probe set comprising the 6 oligonucleotides of SEQ ID NO: 1-6. Also preferred is that SEQ ID NOS: 5-6 are labeled with FAM/BHQ-1. In a further preferred embodiment, steps (c) and (d) are performed in a SmartCycler. In another preferred embodiment, the method further comprises use of internal control primers and probes.

The invention is also directed to a method of detecting the presence or absence of respiratory syncytial virus (RSV) in a nucleic acid sample suitable for use in a real-time reverse transcription polymerase chain reaction (RT PCR) assay comprising the steps of: (a) forming a RT PCR solution containing at least a portion of said nucleic acid sample, a RT PCR primer and probe set comprising one or more oligonucleotides having the sequences set forth in SEQ ID NOS: 1-6, a mixture of nucleoside triphosphate monomers, and an enzyme Taq polymerase in a buffered solution, (b) carrying out a RT PCR reaction on the solution formed in step (a); and (c) detecting the presence or absence of RT-PCR products formed from said solution, wherein the presence of RT-PCR products formed by the amplification of RSV nucleic acid indicates the presence of RSV in the sample and the absence of said RT-PCR products indicates the absence of RSV in the sample. In a preferred embodiment, the method uses a primer and probe set comprising the 6 oligonucleotides of SEQ ID NO: 1-6. Also preferred is that SEQ ID NOS: 5-6 are labeled with FAM/BHQ-1. In a further preferred embodiment, steps (b) and (c) are performed in a SmartCycler. In another preferred embodiment, the method further comprises use of internal control primers and probes.

The invention is also directed to a kit for the detection of respiratory syncytial virus in a sample, said kit comprising one or more oligonucleotide primers and probes comprising the sequences set forth in SEQ ID NOS: 1-6. In a preferred embodiment, the kit comprises the 6 oligonucleotide primers and probes comprising the sequences set forth in SEQ ID NOS: 1-6. Kits of the present invention may further comprise an internal control.

Definitions

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is a N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

The term “primer” or “oligonucleotide primer” as used herein, refers to an oligonucleotide which acts to initiate synthesis of a complementary DNA strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer is first treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “probe” or “oligonucleotide probe” refers to a structure comprised of a polynucleotide, as defined above that contains a nucleic acid sequence complementary to a nucleic acid sequence present in the target nucleic acid analyte. The polynucleotide regions of probes may be composed of DNA, and/or RNA, and/or synthetic nucleotide analogs. When an “oligonucleotide probe” is to be used in a 5′ nuclease assay, such as the TaqMan™ technique, the probe will contain at least one fluorescer and at least one quencher which are digested by the 5′ endonuclease activity of a polymerase used in the reaction in order to detect any amplified target oligonucleotide sequences. In this context, the oligonucleotide probe will have a sufficient number of phosphodiester linkages adjacent to its 5′ end so that the 5′ to 3′ nuclease activity employed can efficiently degrade the bound probe to separate the fluorescers and quenchers.

As used herein, a “biological sample” refers to a sample of tissue or fluid isolated from a subject that commonly includes nucleic acids from the subject. Typical samples that include nucleic acids are known in the art and include but are not limited to, blood, plasma, serum, fecal matter, urine, bone marrow, bile, spinal fluid, lymph fluid, samples of the skin, secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

Respiratory Syncytial Virus

RSV is a nonsegmented negative strand RNA virus of Order Mononegavirales. The mononegaviruses constitute a large and diverse Order that includes four families: Family Rhabdoviridae, represented by vesicular stomatitis virus (VSV) and rabies virus; Family Bomaviridae, represented by boma disease virus; Family Filoviridae, represented by Marburg and Ebola viruses, and Family Paramyxoviridae. This latter family is further divided into two subfamilies: Paramyxovirinae, which includes Sendai, measles, mumps and parainfluenza viruses, and Pneumovirinae, which includes respiratory syncytial virus.

The genome of a mononegavirus is a single strand of RNA that contains from 5 (VSV) to 11 (RSV) genes arranged in a linear array. The mononegavirus genome does not encode protein directly (hence the designation “negative sense”), but rather encodes complementary positive-sense mRNAs that each encode one or more proteins. Typically, a gene begins with a short gene-start signal and ends with a short gene-end signal. These signals usually consist of 8 to 12 nucleotides and usually are highly conserved between genes of a given virus and to a lesser extent between related viruses.

In the case of RSV, the genome is more than 15.2 kb in length and is transcribed into 10 separate major mRNAs that encode 11 identified proteins. Specifically, the RSV gene order is 3′-NS1-NS2-N-P-M-SH-G-F-M2-L-5′, and the M2 mRNA encodes two proteins, M2-1 and M2-2 from overlapping open reading frames (ORFs). The gene-start and gene-end signals of RSV, together with sequences involved in RNA replication and in promoter function, also have been identified and analyzed in ongoing work (Bukreyev et al., J. Virol. 70:6634-6641, 1996; Collins et al., Proc. Natl. Acad. Sci. USA 88:9663-9667, 1991; Mink et al., Virology 185:615-624, 1991; Grosfeld et al., J. Virol. 69:5677-5686, 1995; Hardy and Wertz, J. Virol. 72:520-526, 1998; Hardy et al., J. Virol. 73:170-176, 1999; Kuo et al., J. Virol. 70:6143-6150, 1996; Kuo et al., J. Virol. 70:6892-6901, 1996; Samal and Collins, J. Virol. 70:5075-5082, 1996; Kuo et al., J. Virol. 71:4944-4953, 1997; Feams and Collins, J. Virol. 73:388-397, 1999).

The 3′ end of a mononegavirus genome contains a promoter that directs entry of the polymerase (Lamb and Kolakofsky, Fields Virology, 1:1177-1204, 1996; and Wagner and Rose, Fields Virology, 1121-1136, 1996). This promoter is contained completely or in part in an extragenic leader region at the 3′ end of the genome. The polymerase then transcribes the genome 3′-to-5′ in a linear, stop-restart manner guided by the gene-start and gene-end signals. The gene-start signal of each gene directs initiation of the synthesis of the corresponding mRNA and the gene-end signal directs polyadenylation, termination and release of the corresponding mRNA. The polymerase then remains template-bound and reinitiates at the next downstream gene-start signal. This process is repeated to transcribe one gene after another in their 3′-to-5′ order (Lamb and Kolakofsky, Fields Virology 1:1177-1204, 1996; Wagner and Rose, Fields Virology, 1121-1136, 1996; Abraham and Banedjee, Proc. Natl. Acad. Sci. USA 73:1504-1508, 1976; Ball and White, Proc. Natl. Acad. Sci. USA, 73:442-446, 1976; Ball, J. Virol. 21:411-414, 1977; Banedjee et al., J. Gen. Virol. 34:1-8, 1977; Iverson and Rose, Cell 23:477-484, 1981; Iverson and Rose, J. Virol. 44:356-365, 1982; Banedjee et al., Pharmacol. Ther. 51:47-70, 1991).

Four of the RSV proteins enumerated above are nucleocapsid polymerase proteins, namely the major nucleocapsid N protein, the phosphoprotein P, and polymerase protein L, and the transcription antitermination protein M2-1. Three are surface glycoproteins, namely the attachment G protein, the fusion F glycoprotein responsible for penetration and syncytium formation, and the small hydrophobic SH protein of unknown function. The matrix M protein is an internal virion protein involved in virion formation. There are two nonstructural proteins NS 1 and NS2 of unknown function. Finally, there is an ORF in the M2 mRNA which encodes an RNA regulatory factor M2-2.

As used herein, “RSV gene” generally refers to a portion of the RSV genome encoding an mRNA and typically begins at the upstream end with the 10-nucleotide gene-start (GS) signal and ends at the downstream end with the 12 to 13-nucleotide gene-end (GE) signal. Ten such genes for use within the invention are known for RSV, namely NS1, NS2, N, P, M, SH, G, F, M2 and L. The term “gene” is also used herein to refer to a “translational open reading frame” (ORF). ORF is more specifically defined as a translational open reading frame encoding a significant RSV protein, of which 11 are currently recognized: NS1, NS2, N, P, M, SH, G, F, M2-1 (alternatively, M2(ORF1)), M2-2 (alternatively, M2(ORF2)), and L. Thus, the term “gene” interchangeably refers to a genomic RNA sequence that encodes a subgenomic RNA, and to an ORF (the latter term applies particularly in a situation such as in the case of the RSV M2 gene, where a single mRNA contains two overlapping ORFs that encode distinct proteins). Collins et al., J. Gen. Virol. 71:3015-3020, 1990; Bermingham and Collins, Proc. Natl. Acad. Sci. USA 96:11259-11264, 1999; Ahmadian et al., EMBO J. 19:2681-2689, 2000; Jin et al., J. Virol. 74:74-82, 2000.

The present invention utilizes sequences from the N gene, which is one of the most conserved genes in the RSV genome. Sequences from this gene were selected based on lack of homology with other prokaryotic and eukaryotic nucleotide sequences. The optimal sequences utilized were determined based upon experimentation.

PCR Techniques

PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818.

RNAs may be amplified by reverse transcribing the mRNA into cDNA, and then performing PCR (RT-PCR), as described above. Alternatively, a single enzyme may be used for both steps as described in U.S. Pat. No. 5,322,770. mRNA may also be reverse transcribed into cDNA, followed by asymmetric gap ligase chain reaction (RT-AGLCR) as described by Marshall et al. (1994) PCR Meth. App. 4:80-84.

Reverse transcriptase (RT)-real time PCR (RT-PCR) has also been described (Gibson et al. Genome Methods, 6: 995-1001 (1996)). The Sequence Detection system (ABI Prism, ABD of Perkin Elmer, Foster City, Calif.) uses a 96-well thermal cycler that can monitor fluorescent spectra in each well continuously in the PCR reaction; therefore the accumulation of PCR product can be monitored in ‘real time’ without the risk of amplicon contamination of the laboratory.

A “PCR solution” refers to a solution containing the reagents considered essential to PCR, namely primers for the target nucleic acid, a thermostable DNA polymerase, a DNA polymerase cofactor, and one or more deoxyribonucleoside-5′-triphosphates. Other optional reagents and materials used in PCR are described below.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens) and the like. The term “fluorescer” refers to a substance or a portion thereof which is capable of exhibiting fluorescence in the detectable range.

The fluorogenic 5′ nuclease assay, known as the TaqMan™ assay (Perkin-Elmer), is a powerful and versatile PCR-based detection system for nucleic acid targets. Hence, primers and probes derived from regions of the RSV genome described herein can be used in TaqMan™ analyses to detect the presence of infection in a biological sample. Analysis is performed in conjunction with thermal cycling by monitoring the generation of fluorescence signals. The assay system dispenses with the need for gel electrophoretic analysis, and has the capability to generate quantitative data allowing the determination of target copy numbers.

The fluorogenic 5′ nuclease assay is conveniently performed using, for example, AmpliTaq Gold™ DNA polymerase, which has endogenous 5′ nuclease activity, to digest an internal oligonucleotide probe labeled with both a fluorescent reporter dye and a quencher (see, Holland et al. Proc. Natl. Acad. Sci. USA 88:7276-7280 (1991); and Lee et al. Nucl. Acids Res. 21:3761-3766 (1993)). Assay results are detected by measuring changes in fluorescence that occur during the amplification cycle as the fluorescent probe is digested, uncoupling the dye and quencher labels and causing an increase in the fluorescent signal that is proportional to the amplification of target DNA. A typical fluorophore is carboxyfluorescein (FAM).

The amplification products can be detected in solution or using solid supports. The TaqMan™ probe is designed to hybridize to a target sequence within the desired PCR product. The 5′ end of the TaqMan™ probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. Excision of the 5′ fluorophore results in an increase in fluorescence which can be detected.

The quencher can be any material that can quench at least one fluorescence emission from an excited fluorophore being used in the assay. A number of suitable quenchers are known in the art and are commercially available. Typical quenchers include DABCYL, BHQ-1, BHQ-2, BHQ-3, a metal nanoparticle, and a semiconductor nanocrystal having a broad absorbance spectra and an emission wavelength outside the range being detected in the current assay, or a semiconductor nanocrystal having no detectable emission.

Detection devices are available for measuring changes in fluorescence emission intensity during amplification according to the present invention. Illustrative of such devices are: the ABI 7700 of Applied Biosystems; the BDProbeTecET fluorescent reader of Becton Dickinson Microbiology Systems (Sparks, Md.), see Little et al., Clin. Chem. 45: 777-784 (1999); and the “SmartCycler” of Cepheid (Sunnyvale, Calif.). This measurement is done in real-time, i.e., in the same time frame that the amplification product accumulates in the reaction. Other methods are available for gauging changes in fluorescence which result from probe digestion. Exemplary of such alternative techniques is fluorescence polarization, as described, for example, in U.S. Pat. No. 5,593,867. Fluorescence polarization distinguishes larger molecules from smaller molecules based on different rates of molecular tumbling

In certain embodiments, an internal control (IC) or an internal standard is added to serve as a control for target capture and amplification. Preferably, the IC includes a sequence that differs from the target sequence, is capable of hybridizing with the probe sequences used for separating the oligonucleotides specific for the organism from the sample, and is capable of amplification. The use of the IC permits the control of the separation process, the amplification process, and the detection system, and permits the monitoring of assay performance and quantification for the sample(s).

The above-described assay reagents, including the primers, probes, solid support with bound probes, as well as other detection reagents, can be provided in kits, with suitable instructions and other necessary reagents, in order to conduct the assays as described above. The kit will normally contain in separate containers the combination of primers and probes (either already bound to a solid matrix or separate with reagents for binding them to the matrix), control formulations (positive and/or negative), labeled reagents when required by the assay format and signal generating reagents (e.g., enzyme substrate) if the label does not generate a signal directly. Instructions (e.g., written, tape, VCR, CD-ROM, etc.) for carrying out the assay usually will be included in the kit. The kit can also contain, depending on the particular assay used, other packaged reagents and materials (i.e. wash buffers and the like). Standard assays, such as those described above, can be conducted using these kits.

The following nonlimiting examples are provided to further illustrate the present invention.

### EXAMPLES

### Example 1

### Detection of RSV by RT-PCR Using Optimized Primers and Probes Primers and TaqMan Probes

The assay employs two sets of primers and probes, detecting both viral subgroups in a single reaction. Primers and probes show homology to all eleven strain A sequences and all sixteen strain B sequences found in GenBank as of this time. Probes are labeled with FAM/BHQ-1.

Nucleotide sequences of Primers and Probe for Detection of RSV A

| | |
|---|---|
| Primers: | |
| 5′TACACTCAACAAAGATCAACTTCTGTCA | (SEQ ID NO: 1) |
| (28mer) Tm = 59° C. | |
| 5′CATGCCACATAACTTATTGATGTGTTT | (SEQ ID NO: 2) |
| (27mer) Tm = 59° C. | |
| Probe: | |
| 5′CACCATCCAACGGAGCACAGGAGA | (SEQ ID NO: 5) |
| (24mer) Tm = 68° C. | |

Amplicon: 1 tacactcaac aaagatcaac ttctgtcatc cagcaaatac accatccaac ggagcacagg agatagtatt gatactccta affatgatgt gcagaaacac atcaataagt tatgtggcat g 121 (SEQ ID NO:7)

Nucleotide Sequences of Primers and Probe for Detection of RSV B

| | |
|---|---|
| Primers: | |
| 5′CATTAAATAAGGATCAGCTGCTGTCA | (SEQ ID NO: 3) |
| (26mer) Tm = 60° C. | |
| 5′GCATACCACATAGTTTGTTTAGGTGTTT | (SEQ ID NO: 4) |
| (28mer) Tm = 59° C. | |
| Probe: | |
| 5′ATAATATTGACAC TCCCAATTAT GATGTGC | (SEQ ID NO: 6) |
| (30mer) Tₘ = 69° C. | |

Amplicon: 1 catta aataaggatc agctgctgtc atccagcaaa tacactattc aacgtagtac aggagataat attgacactc ccaattatga tgtgcaaaaa cacctaaaca aactatgtgg tatgc 120 (SEQ ID NO:8)

Protocol for Detection of RSV (One Step RT-PCR)

Master Mix (Qiagen OneStep RT-PCR Kit):

The master mixture contains following reagents:

One time 5× buffer

One μl of enzyme solution/25 μl reaction

dNTPs: 0.4 mM

CIC forward primer: 0.2 μM

CIC reverse primer: 0.4 μM

CIC probe: 0.2 μM

RsvA forward primer: 0.2 μM

RsvA reverse primer: 0.4 μM

RsvA probe: 0.1 μM

RsvB forward primer: 0.2 μM

RsvB reverse primer: 0.4 μM

RsvB probe: 0.15 μM

Rnsine: 5 units

Five μl of sample

RT-PCR Protocol Using Cepheid SmartCycler II:

Reverse transcription: at 48° C. for 900 seconds

Activation of Taq DNA polymerase: at 95° C. for 900 seconds

PCR (50 cycles): at 95° C. for 15 seconds; followed at 60° C. for 30 seconds with Optics ON and 72° C. for 15 seconds.

Rsv strains tested during evaluation of this protocol include Rsv Strain A-2 (Advanced Biotechnologies, Inc.), Rsv A Strain Long and Rsv B Strain B WV/14617/85 (ATCC). RSV A viral particle (Rsv Strain A-2, Advanced Biotechnologies, Inc) and Rsv A and B viral lysates (ATCC) were extracted using Viral RNA Mini kit (Qiagen) or MiniMag (Biomerieux, INC. Durham, N.C., USA) according to manufacturer instructions. RT-PCR was performed using QIAGEN OneStep RT-PCR kit and SmartCycler II (Cepheid, Calif.).

Specificity of the detection was examined through researching DNA sequence database and experimentally. A FASTA nucleotide sequence similarity search (GenBank and EMBL) revealed no significant homologous targets in both prokaryotic and eukaryotic nucleotide sequence database. No cross-reactivity was noticed with other tested respiratory viruses including Adv5, EBV, SV40, HSV types 1 and 2, HHV 6 and 8, and human CMV.

Sensitivity was demonstrated to<100 copies of viral genome/ml sample for RSV subgroup A and to<10 TCID₅₀/ml for RSV subgroup B.

Quantitative linearity was demonstrated to be from 10² to 10⁶ copies of viral genome.

Various publications, patent applications and patents are cited herein, the disclosures of which are incorporated by reference in their entireties.

We claim:

1. An oligonucleotide primer optimized for the detection of respiratory syncytial virus (RSV) comprising a sequence selected from the group consisting of SEQ ID NOS: 1, 2, 3 and 4.

2. The oligonucleotide primer of claim 1 comprising the sequence set forth in SEQ ID NO. 1.

3. The oligonucleotide primer of claim 1 comprising the sequence set forth in SEQ ID NO. 2.

4. The oligonucleotide primer of claim 1 comprising the sequence set forth in SEQ ID NO. 3.

5. The oligonucleotide primer of claim 1 comprising the sequence set forth in SEQ ID NO. 4.

6. An oligonucleotide probe optimized for the detection of respiratory syncytial virus (RSV) comprising a sequence selected from the group consisting of SEQ ID NOS: 5 and 6.

7. The oligonucleotide probe of claim 6 comprising the sequence set forth in SEQ ID NO. 5.

8. The oligonucleotide probe of claim 6 comprising the sequence set forth in SEQ ID NO. 6.

9. A real-time reverse transcription polymerase chain reaction (RT PCR) primer and probe set specific for respiratory syncytial virus (RSV), said primer and probe set comprising one or more oligonucleotide primers of claim 1 or a probe comprising SEQ ID NOS: 5 and 6.

10. The real-time reverse transcription polymerase chain reaction (RT PCR) primer and probe set specific of claim 9 wherein said primer and probe set comprises the sequences set forth in SEQ ID NOS: 1, 2, 3, 4, 5 and 6.

11. A method of detecting the presence or absence of respiratory syncytial virus (RSV) in a patient, said method comprising the steps of:
a) preparing a nucleic acid sample from the patient suitable for use in a real-time reverse transcription polymerase chain reaction (RT PCR) assay;
b) forming a RT PCR solution containing at least a portion of said nucleic acid sample from step (a), a RT PCR primer and probe set comprising one or more oligonucleotide primers of claim 1 or a probe comprising SEQ ID NOS: 5 or 6, a mixture of nucleoside triphosphate monomers, and an enzyme Taq polymerase in a buffered solution;
c) carrying out a RT PCR reaction on the solution formed in step (b); and
d) detecting the presence or absence of RT-PCR products formed from said solution,
wherein the presence of formed RT-PCR products indicates the presence of RSV in the patient and the absence of formed RT-PCR products indicates the absence of RSV in the patient.

12. The method of claim 11 wherein the RT-PCR primer and probe set comprises the sequences set forth in SEQ ID NO: 1, 2, 3, 4, 5, and 6.

13. The method of claim 11 wherein SEQ ID NO: 5 and 6 are labeled with FAM/BHQ-1.

14. The method of claim 10 wherein steps (c) and (d) are performed in a SmartCycler.

15. The method of claim 10 further comprising monitoring assay performance via internal control primers and probes.

16. A method of detecting the presence or absence of respiratory syncytial virus (RSV) in a nucleic acid sample suitable for use in a real-time reverse transcription polymerase chain reaction (RT PCR) assay comprising the steps of:
a) forming a RT PCR solution containing at least a portion of said nucleic acid sample, a RT PCR primer and probe set comprising one or more oligonucleotide primers of claim 1 or a probe comprising SEQ ID NOS: 5 or 6, a mixture of nucleoside triphosphate monomers, and an enzyme Taq polymerase in a buffered solution;
b) carrying out a RT PCR reaction on the solution formed in step (a); and
c) detecting the presence or absence of RT-PCR products formed from said solution,
wherein the presence of RT-PCR products formed by the amplification of RSV nucleic acid indicates the presence of RSV in the sample and the absence of said RT-PCR products indicates the absence of RSV in the sample.

17. The method of claim 16 wherein the RT-PCR primer and probe set comprises the sequences set forth in SEQ ID NO: 1, 2, 3, 4, 5, and 6.

18. A kit for the detection of respiratory syncytial virus in a sample, said kit comprising one or more oligonucleotide primers and probes comprising the sequences set forth in SEQ ID NOS: 1, 2, 3, 4, 5 and 6.

19. The kit of claim 18 comprising the 6 oligonucleotide primers and probes comprising the sequences set forth in SEQ ID NOS: 1, 2, 3, 4, 5, and 6.

20. The kit of claim 18 further comprising an internal control.
